(19) United States
(12) Patent Application Publication
(10) Pub. No.: US 2024/0062308 A1
(43) Pub. Date: Feb. 22, 2024

(54) DATA AND DOCUMENT MANAGEMENT FOR MEDICAL SERVICE DURATIONS AND OTHER APPLICATIONS

(71) Applicant: Adam In Claims LLC, Dorchester, MA (US)

(72) Inventors: Adam W Rock, Dorchester, MA (US); Todd W Elvers, Columbia, MO (US); Hunter Scheib, Columbia, MO (US)

(21) Appl. No.: 18/235,650

(22) Filed: Aug. 18, 2023

Related U.S. Application Data
(60) Provisional application No. 63/399,145, filed on Aug. 18, 2022.

(51) Int. Cl. G06Q 40/08; G16H 15/00

(52) U.S. Cl. CPC G06Q 40/08; G16H 15/00

(57) ABSTRACT

A medical claim procedure processing system includes application software configured for creating accounts and obtaining protocols for new clients. A master database is loaded with client data, including claim details. Data input is obtained from medical service provider bills. A claim dashboard displays the data from searched claims, including claim summaries. The system generates a medical service occurrence purview. A medical claim procedure processing method includes the steps of creating accounts for new clients, obtaining protocols, loading a master database with client data, setting up claims, displaying a claim dashboard and generating a medical service occurrence purview.

### CROSS-REFERENCE TO RELATED APPLICATION

This application claims priority in U.S. Provisional Patent Application No. 63/399,145, filed Aug. 18, 2022, which is incorporated herein by reference.

### BACKGROUND OF THE INVENTION

### 1. Field of the Invention

The present invention relates generally to data and document management, and in particular to a system and method for benchmarking, predictive analytics and reporting medical service durations, which are relevant to Official Disability Guidelines (ODG)¹, and other evidence-based disability guideline applications. ¹ ODG is a nation-wide database of workers' compensation and occupational health injuries owned by the Hearst Health Network and administered by its MCG Health division.

### 2. Description of the Related Art

Data and document management products and services currently utilize solutions ranging from local area networks (LANs) to cloud-based software solutions. Current systems for managing claims in the workers compensation and healthcare fields include: Guidewire; Duck Creek Technologies; Ventiv Technology Records Retrieval: Ontellus—SmartChron™; and Sapiens ClaimsPro.

Heretofore there has not been available a system or method for managing data and documents with the advantages and features of the present invention.

### SUMMARY OF THE INVENTION

The present invention generally provides a cloud-based system and method for managing data and documents with indexed medical services providing service durations for providers, treatments, and diagnoses.

### BRIEF DESCRIPTION OF THE DRAWINGS

The drawings constitute a part of this specification and include exemplary embodiments of the present invention illustrating various objects and features thereof.

FIG. 1 shows a claim summary occurrence purview with multiple fields for claim specific data input, including providers, diagnoses, treatments and medical events.

FIG. 2 is a flowchart showing an example of a medical claim procedure embodying an exemplary aspect of the present invention. The output includes a medical service occurrence purview generated by the system and method of the present invention.

FIG. 3 shows a medical institution bill generated by the system and method of the present invention.

FIG. 4 shows a medical physician bill generated by the system and method of the present invention.

### DETAILED DESCRIPTION OF THE PREFERRED EMBODIMENTS

### I. Introduction and Environment

As required, detailed aspects of the present invention are disclosed herein, however, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art how to variously employ the present invention in virtually any appropriately detailed structure.

Certain terminology will be used in the following description for convenience in reference only and will not be limiting. Said terminology will include the words specifically mentioned, derivatives thereof and words of similar meaning.

### II. Preferred Embodiment

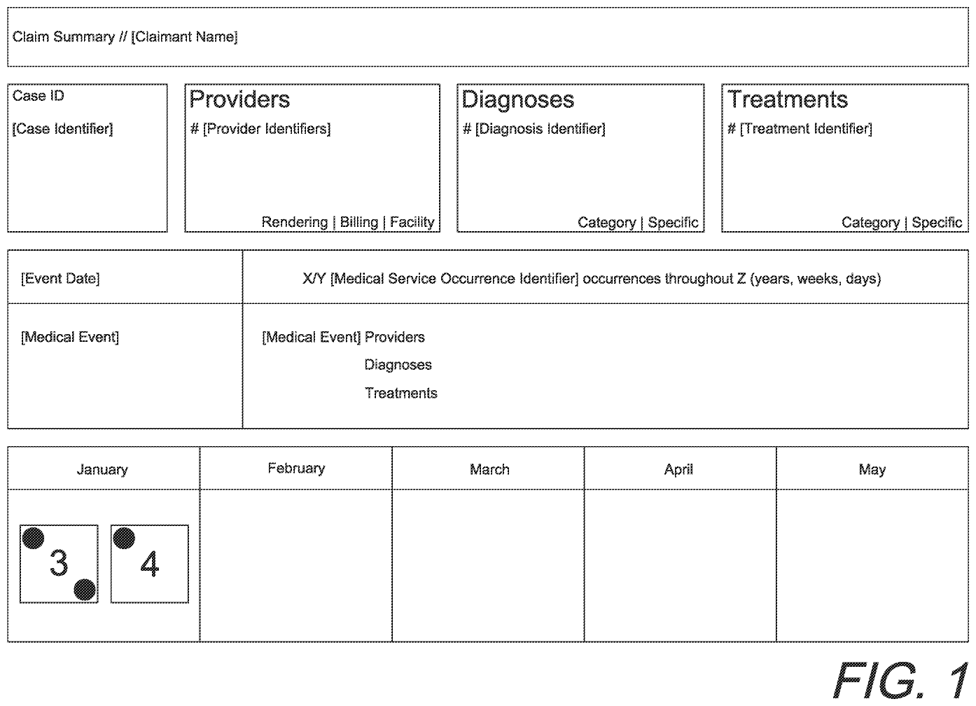

Referring to the drawings in more detail, FIGS. 1-4 show an exemplary aspect of the present invention. FIG. 1 shows a claim summary occurrence purview with multiple fields for claim specific data input, including providers, diagnoses, treatments and medical events.

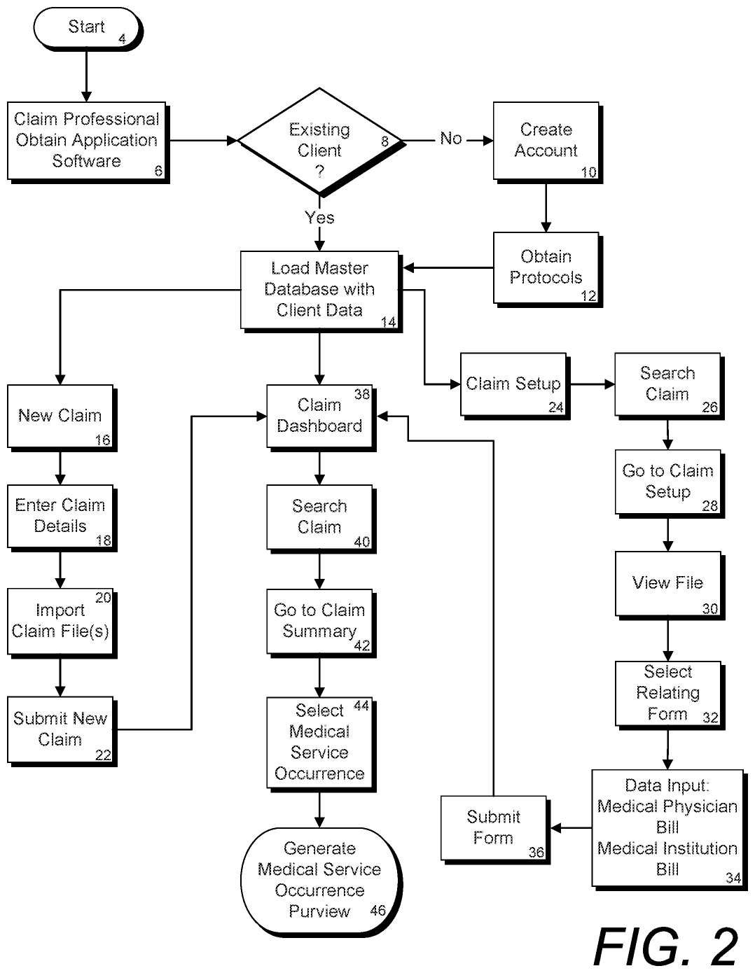

FIG. 2 is a flowchart showing an example of a medical claim procedure 2 embodying an exemplary aspect of the present invention. The output includes a medical service occurrence purview generated by the system and method of the present invention.

From a start 4 the method proceeds to a Claim Professional Obtain Application Software step 6. At Existing Client? decision box 8, “No” leads to Create Account step 10, Obtain Protocols 12 and Load Master Database with Client Data 14. A Yes decision at 8 leads directly to the Load Master Database with Client Data 14 step.

New Claim 16 leads to Enter Claim Details 18, Import Claim File(s) 20 and Submit New Claim 22. From the Load Master Database step 14, the procedure 2 activates a Claim Setup 24, which leads to Search Claim 26, Go to Claim Setup 28, View File 30, Select Relating Form 32, Data Input: Medical Physician Bill and Medical Institution Bill 34, Submit Form 36 and back to the Claim Dashboard 38.

Also from Load Master Database 14, the procedure 2 activates Claim Dashboard 38, Search Claim 40, Go to Claim Summary 42, Select Medical Service Occurrence 44 and Generate Medical Service Occurrence Purview 46.

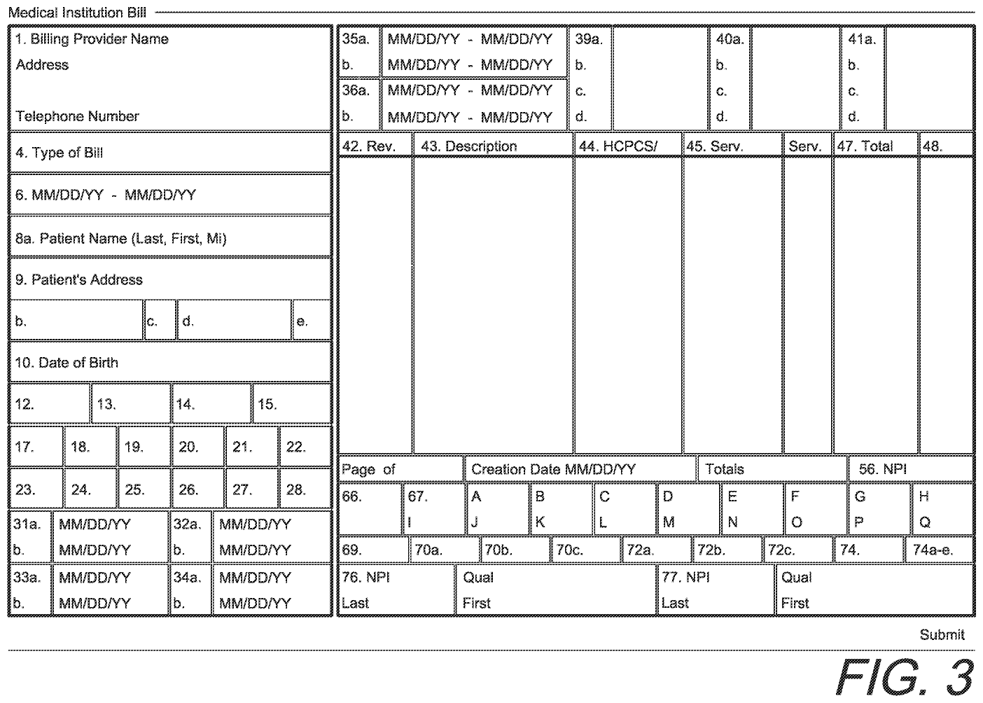

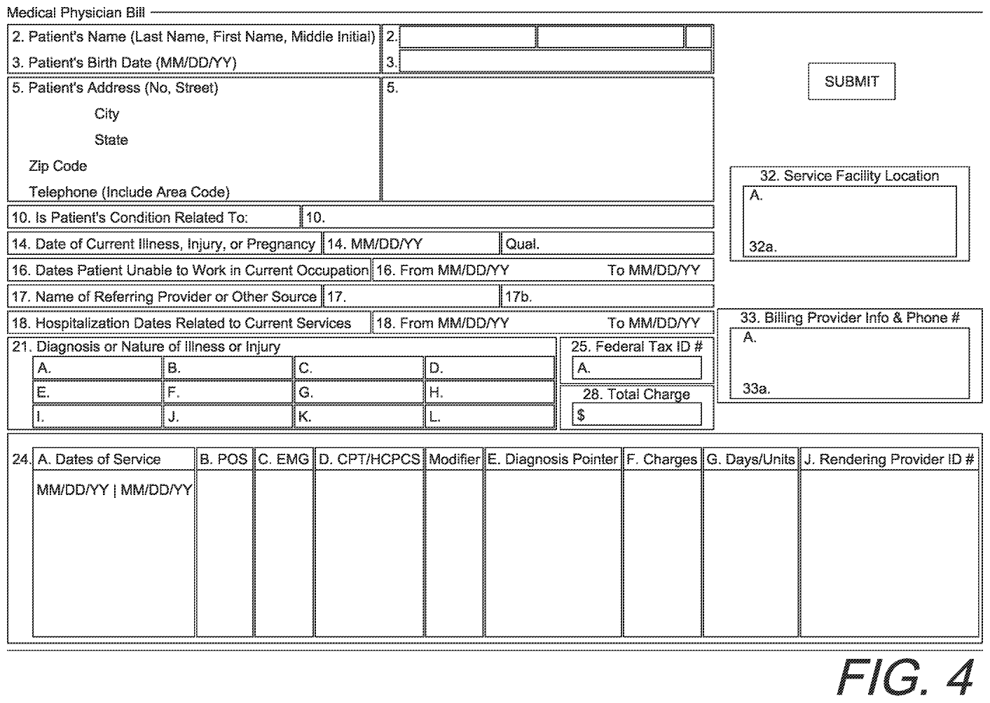

FIG. 3 shows a medical institution bill generated by the system and method of the present invention. FIG. 4 shows a medical physician bill generated by the system and method of the present invention.

It is to be understood that while certain embodiments and/or aspects of the invention have been shown and described, the invention is not limited thereto and encompasses various other embodiments and aspects. For example, and without limitation on the generality of useful applications of the present invention, other potential data and document management applications include a wide range of claims for various occurrences and other commercial transactions.

Having thus described the invention, what is claimed as new and desired to be secured by Letters Patent is:

1. A medical claim procedure system comprising:
a claim professional software application configured for identifying an existing client and creating an account for a new client;
the account creation including obtaining protocols and loading a master database with client data;
a new claim, including claim details, importing claim files and submitting the new claim;
a claim set up including searching the claim, going to claim setup viewing a file, selecting a relating form, receiving data input comprising a medical physician bill and a medical institution bill and submitting the form; and
a claim dashboard configured for searching a claim, going to a claim summary selecting a medical service occurrence, generating a medical service occurrence purview.

2. The medical claim procedure system according to claim 1, which includes:
a claim dashboard configured for receiving claim details, client data, account data and protocols.

3. A method of processing a medical claim, which method comprises the steps of:
obtaining claim professional application software;
determining if the claim is for an existing client: if yes, loading a master database with client data; if no, creating an account, obtaining protocols and loading the master database with data;
if a new claim, entering claim details, importing claim files and submitting the new claim;
from the loading master database step, setting up a claim, search and claim, going to claim setup, viewing the file, selecting a relating form, obtaining data input comprising a medical physician bill and a medical institution bill; submitting form and viewing the file; and
from the loading master database step, displaying a claim dashboard, searching a claim, going to a claim summary, selecting a medical service occurrence and generating a medical service occurrence purview.

4. The medical claim procedure processing method according to claim 3, which includes the additional step of:
providing a claim dashboard configured for receiving claim details, client data, account data and protocols.
